(19) United States
(12) Patent Application Publication
(10) Pub. No.: US 2024/0245408 A1
(43) Pub. Date: Jul. 25, 2024

(54) TOOL HOLDER FOR MODULAR TOOL

(71) Applicant: OSTIUM GROUP, SAINT ETIENNE DE MONTLUC (FR)

(72) Inventors: Edgard SOQUENNE, SAINT ETIENNE DE MONTLUC (FR); Vincent RETAILLEAU, SAINT ETIENNE DE MONTLUC (FR); Leopold BIBARD, SAINT ETIENNE DE MONTLUC (FR); Corentin LE HENANF, SAINT ETIENNE DE MONTLUC (FR)

(21) Appl. No.: 18/424,726

(22) Filed: Jan. 26, 2024

Related U.S. Application Data
(63) Continuation of application No. PCT/EP2022/071267, filed on Jul. 28, 2022.

(30) Foreign Application Priority Data
Jul. 28, 2021 (FR) 2108212

(51) Int. Cl. A61B 17/16; A61B 17/00; B25G 3/08; B25G 3/12

(52) U.S. Cl. CPC A61B 17/1604; B25G 3/08; B25G 3/12; A61B 2017/0046

(57) ABSTRACT

A tool holder for a modular tool in which a tool element is trapped between two slidably mating parts, which may be slidingly aligned to trap the tool element, and slidingly separated to free the tool. The sliding operation of the slidably mating parts is defined by mutually engaging retaining elements configured to allow relative movement of said slidably mating parts along said longitudinal axis, but prevent relative movement in any other direction. The tool holder may form part of a kit comprising one or more tool holders, one or more tool elements, and one or more handles. The handle may be comprised with key elements engaging a proximal extremity of the slidably mating parts when aligned, to that with the handle in position, the slidably mating parts cannot be separated, and the tool element released.

### FIELD OF THE INVENTION

The present invention relates to modular tools, and in particular a tool holder element for such tools.

### BACKGROUND OF THE INVENTION

The concept of modularity in tools is almost as old as tool use itself. Many ancient tool designs such as hammers, axes, picks and the like comprise a working element fixed to a handle by some means which allows the periodic replacement of one element or the other. Developments in manufacturing processes over the last two hundred years meanwhile have made it possible to develop releasable fixing mechanisms. For example, screw driver heads are commonly available as 6.35 mm (quarter inch) hexagon bits, which may be inserted as required in a standard handle. The bits may be secured magnetically, or by means of a spring loaded ball bearing engaging a groove in the bit. In some contexts meanwhile, the operational constraints extant in that context may tend to lead away from such approaches. In the field of surgical instruments for example, the high forces involved along with sterilisation requirements may tend to indicate an all-in-one approach.

FIG. 1 shows an osteotome as known in the state of the art.

As shown in FIG. 1, a box osteotome 100 comprises a handle 102 with a strike head 101 and a chisel working surface 103. The osteotome 100 is typically made entirely of stainless steel.

Notwithstanding the foregoing, the cost of manufacturing a complete set of stand-alone tools for example as shown in FIG. 1 can be considerable, and even in the field of surgical tools, some attempts at modular tools are known.

In this, as in any field where a solid positive engagement between the handle and further elements is a critical requirement, special consideration must be given to the securing mechanism.

FIG. 2 shows a releasable securing mechanism for modular tools as known in the state of the art.

FIG. 2 shows a conventional securing mechanism known in the art as a “Hudson Fitting”. In particular, FIG. 2 shows a male Hudson fitting 200, attached to a tool element 201. The fitting comprises a cylindrical member 203 with a semi-circular channel or groove 204. When the cylindrical member 203 is slid into the corresponding female element, a spring loaded ball bearing 205 engages the channel so as to prevent accidental decoupling. In implementations where uncoupling must be avoided in the presence of a separating force, the ball bearing may be replaced with a removable cotter pin or the like. As shown, the fitting also comprises a flattened flange 202 at the proximal end of the fitting closest to the tool element 201. The flats of this flange may engage corresponding surfaces on the corresponding female element when the coupling is fully inserted, so that rotational forces may be effectively transferred between the two elements of the coupling.

Fittings such as that shown in FIG. 1 have been found unsatisfactory in terms of their ability to provide a solid positive engagement between the handle and further elements, whilst supporting uncoupling with a minimum of force and dexterity when required, yet averting the risk of accidental uncoupling. As such, it is desired to provide an improved coupling addressing some or all of these concerns.

### SUMMARY OF THE INVENTION

In accordance with the present invention in a first aspect there is provided a tool holder for a modular tool, the tool holder having a distal end and a proximal end disposed on a longitudinal axis, the tool holder being for a modular tool and split longitudinally into two slidably mating parts, the slidably mating parts being provided with mutually engaging retaining elements, the mutually engaging retaining elements being configured to allow relative movement of the slidably mating parts along the longitudinal axis, but prevent relative movement in any other direction, a first slidably mating part being provided with a longitudinal excavation extending from the distal end thereof such that the longitudinal excavation may be obturated but for the distal extremity thereof by sliding the second slidably mating part so as to bring the distal extremity of the second slidably mating part into proximity with the distal extremity of the second slidably mating part, and such that the longitudinal excavation may be exposed by sliding the second slidably mating part so as to separate the distal extremity of the second slidably mating part from the distal extremity of the second slidably mating part, whereby a tool element may be inserted into the excavation when exposed, and trapped therein when obturated.

In accordance with a development of the first aspect, each slidably mating part comprises a respective locking element at a proximal extremity thereof such that when the second slidably mating part is slid so as to bring the distal extremity of the second slidably mating part into proximity with the distal extremity of the second slidably mating part, the respective locking elements align to form a keyed element, whereby the keyed element may be inserted into a corresponding channel of a restraining element so as to prevent further relative sliding movement of the slidably mating parts.

In accordance with a development of the first aspect, the dimensions of respective locking elements vary from front to back so that the force required to slide the keyed element formed thereby into the corresponding keyed channel of a restraining element increases as the keyed element of the tool progresses into the slot.

In accordance with a development of the first aspect, each slidably mating part comprises a respective locking element at a proximal extremity thereof such that when the second slidably mating part is slid so as to bring the distal extremity of the second slidably mating part into proximity with the distal extremity of the first slidably mating part, the respective locking elements align to form a body whose outer extremities conform to a bounding cylinder, the locking elements being provided with curved features such that when the second slidably mating part is slid so as to bring the distal extremity of the second slidably mating part into proximity with the distal extremity of the first slidably mating part, the curved features elements align to define at least part of one or more helical features coaxial with the bounding cylinder, such that a threaded ring may by screwed onto the tool holder.

In accordance with a development of the first aspect, the pitch, the minor diameter, the major diameter, or the root to crest ratio of the external helical features varies along the length of the external helical features so that the force required to rotate the ring increases as the ring is rotated about the distal end of the member from the retracted position to the extended position.

In accordance with the present invention in a second aspect there is provided a modular tool kit comprising a tool holder according to the first aspect, and a handle comprising the keyed channel, whereby the handle constitutes the restraining element so as to prevent further relative sliding movement of the slidably mating parts.

In accordance with a development of the second aspect, the handle comprises a distal extremity whose outer dimensions conform to a bounding cylinder bearing one or more helical features coaxial with the bounding cylinder, such that a threaded ring may by screwed onto the body.

In accordance with a development of the second aspect, the handle comprises a distal extremity whose outer dimensions conform to a bounding cylinder bearing one or more helical features coaxial with the bounding cylinder, the modular tool kit further comprising a threaded ring that may by screwed onto the handle when engaged with the helical features.

In accordance with a development of the second aspect, the helical features permit an angular displacement of the threaded ring when rotated between an extended position and a retracted position is between 170 and 190 degrees.

In accordance with a development of the second aspect, the handle is provided with a second external helical thread parallel the first external helical thread, and wherein the threads of the ring engage both the first external helical thread and the second external helical thread.

In accordance with a development of the second aspect, the threaded ring comprises an indentation at the distal edge thereof, the indentation being positioned on the circumference of the ring such that when the ring is in the retracted position the indentation is aligned with the slot.

In accordance with a development of the second aspect, the modular tool kit further comprises a tool element adapted to be inserted into the excavation when exposed, and trapped therein when obturated.

In accordance with a development of the second aspect, the tool element is adapted for surgical use, whereby a modular tool composed from the modular toolkit constitutes a surgical instrument.

In accordance with a development of the second aspect, the tool element is for surgery of the orthopaedic surgery or bone traumatology.

In accordance with a development of the second aspect, the tool element is an osteotome or implant impactor.

In accordance with a development of the second aspect, the tool holder is composed of a synthetic material or a synthetic composite material.

In accordance with a development of the second aspect, the tool holder is composed of a glass fibre reinforced polyarylamide.

### BRIEF DESCRIPTION OF THE DRAWINGS

The above and other advantages of the present invention will now be described with reference to the accompanying drawings, for illustration purposes only, in which:

FIG. 1 shows an osteotome as known in the state of the art;

FIG. 2 shows a releasable securing mechanism for modular tools as known in the state of the art;

FIG. 3 shows a cross section of tool holder for a modular tool in accordance with an embodiment in a first configuration;

FIG. 4 shows a cross section of tool holder for a modular tool in accordance with embodiment of FIG. 3 in a second configuration;

FIG. 5 presents a three dimensional perspective of one slidably mating part as usable in certain embodiments;

FIG. 6 illustrates a mode of operation of a tool holder in accordance with certain embodiments;

FIG. 7 shows a further development of locking mechanism introduced with reference to FIG. 6;

FIG. 8a shows the ring in a first, retracted position prior to the insertion of the keyed element formed by the respective locking elements 761, 762 of the slidably mating parts forming the tool holder as discussed above;

FIG. 8b shows the handle in a first, retracted position after insertion of the keyed element formed by the respective locking elements 761, 762 of the slidably mating parts forming the tool holder;

FIG. 8c shows the handle in a second, extended position after insertion of the keyed element formed by the respective locking elements 761, 762 of the slidably mating parts forming the tool holder; and

FIG. 9 shows an assembled tool in accordance with an embodiment.

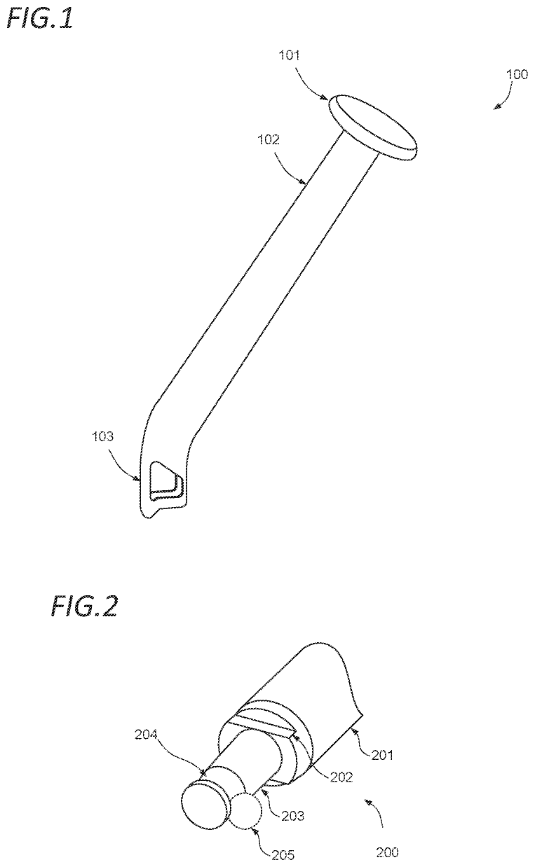

### DETAILED DESCRIPTION

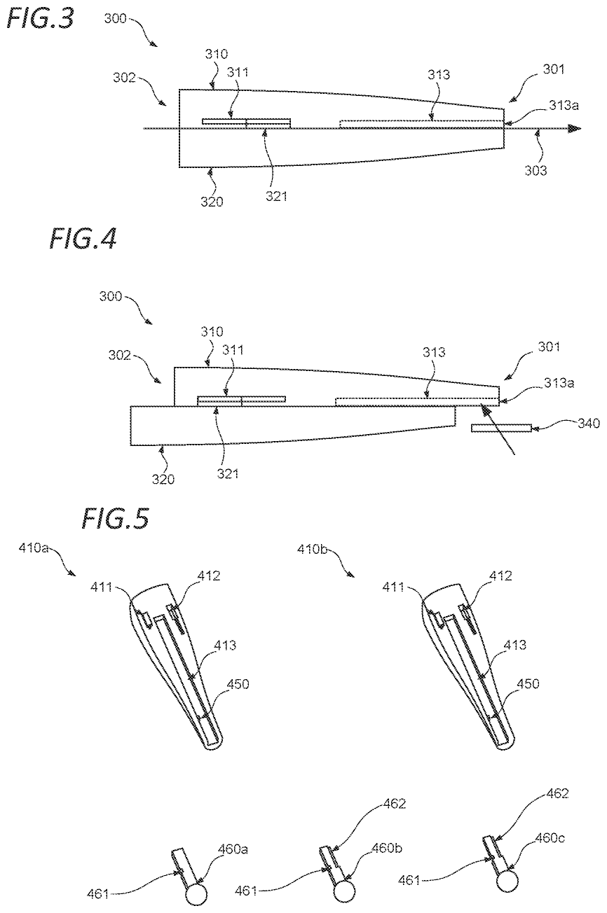

FIG. 3 shows a cross section of tool holder for a modular tool in accordance with an embodiment in a first configuration.

As shown in FIG. 3, a tool holder 300 has a distal end 301 and a proximal end 302 disposed on a longitudinal axis 303, the tool holder for a modular tool being split longitudinally into two slidably mating parts 310 and 320. The slidably mating parts are provided with mutually engaging retaining elements 311, 221 configured to allow relative movement of the slidably mating parts along the longitudinal axis, but prevent relative movement in any other direction.

A first slidably mating part 310 is provided with a longitudinal excavation 313 extending from the distal end 302 such that the longitudinal excavation may in a first configuration as shown in FIG. 3 be obturated but for the distal extremity 313a thereof by sliding the second slidably mating part 320 so as to bring the distal extremity of the second slidably mating part into proximity with the distal extremity of the second slidably mating part.

FIG. 4 shows a cross section of tool holder for a modular tool in accordance with embodiment of FIG. 3 in a second configuration.

FIG. 4 shows elements corresponding to respective elements of FIG. 3 with like reference numerals.

The configuration of FIG. 4 comprises the same elements as described above with respect to FIG. 3 in a second configuration in which the longitudinal excavation 313 is be exposed by sliding the second slidably mating part 320 so as to separate the distal extremity of the second slidably mating part from the distal extremity of the second slidably mating part, whereby a tool 340 element may be inserted into the excavation when exposed, and trapped therein when obturated.

FIG. 5 presents a three dimensional perspective of one slidably mating part as usable in certain embodiments.

As shown in FIG. 5, a slideably mating part 410a similar to those described with reference to FIGS. 3 and 4 is provided. As shown, the slideably mating part is provided with two retaining elements 411, 412, situated either side of the central axis at an equal distance therefrom. The first retaining element 411 has a substantially T shaped cross section, extruded parallel to the axis of the part. The second retaining element 412 is a cavity, narrowing towards the distal end of the part, and dimensioned so that a T shaped retaining element similar to 411 could be inserted at the wider, proximal end, and then slid forward towards the proximal end of the mating part 410a and trapped therein. It will be appreciated that a second slidably mating part, which might conveniently be identical to the slideably mating part 410a, could thus be brought into proximity with the first slideably mating part in an arrangement corresponding to the first configuration presented with reference to FIG. 3 above, and then slid forward so that the two parts are firmly by releasably engaged in the second configuration described above with respect to FIG. 4.

As shown, the excavation 450 is not dimensioned continuously. As shown, there is provided a tooth 45, which may engage a corresponding detent 461 in an insertable tool element 460. By this means, it may be ensured that an inserted tool element cannot move in, or be removed from, the excavation 413 when in the second configuration as described for example with reference to FIG. 4.

Accordingly, certain embodiments may comprise a kit comprising a tool holder as discussed herein, and one or more tool elements adapted to be inserted into the excavation when exposed, and trapped therein when obturated.

Such a tool element may be adapted for surgical use, whereby a modular tool composed from the modular toolkit constitutes a surgical instrument.

Such a tool element may be adapted for orthopaedic surgery or bone traumatology.

Such a tool element may be an osteotome or an implant Impactor . . . .

While as described with respect to FIG. 3 one slidably mating part is provided with an excavation. It will be appreciated with reference to FIG. 5 that it may be advantageous to adopt embodiments based on an identical pair of slidably mating parts, which would not necessarily be operable on the basis of a pair of slidably mating parts and a tool element as shown in FIG. 3, since in such a configuration, the tool 460 could be inserted, but the tooth in the upper slidably mating part (not shown) would run into the tool element and prevent the two mating parts being brought into the second configuration.

One solution to this difficulty is to adapt the shape of the tool element so as to provide an engaging detent on one side only, and configured to clear a tooth on the other side, as shown in tool element 460b, which is provided with a clearing groove 462 on the opposite side to the detent 461. This approach may be followed in such a way that the tool has a “right side” and a “wrong side”, ensuring that the slidingly mating parts can only close on the tool in one orientation, or may be provided with rotationally symmetrical structures as shown with reference to element 460c, which will be operable in either arrangement.

It may be noted that while tool elements 460a, 460b, 460c are shown as having a substantially rectangular cross section which may a prioiri be inserted into the excavation of the slidingly mating part in one of two orientations, other tool formats may be envisaged with different configurations. In particular, the tool element may be of square cross section, offering four possible orientations, or similarly of threefold symmetry (e.g. triangular), six fold symmetry (e.g. hexagonal), of infinite symmetry (e.g. cylindrical), or any other broadly symmetrical configuration.

Another solution is to offset the excavation of the slidably mating part from the central axis as shown with reference to slidingly mating part 410b. In this configuration, a tool part may be dimensioned so as not to extend beyond the excavation of one slidably mating part, but will still be trapped by the second part in the second configuration because the excavation of the second slidingly mating part will be offset with respect to that in which the tool part is situated.

It will be appreciated that the two preceding implementations may be combined. These approaches bring the advantage that only a single, identical slidingly mating part need be manufactured, and two such identical parts brought together to form a tool holder in accordance with embodiments presented above.

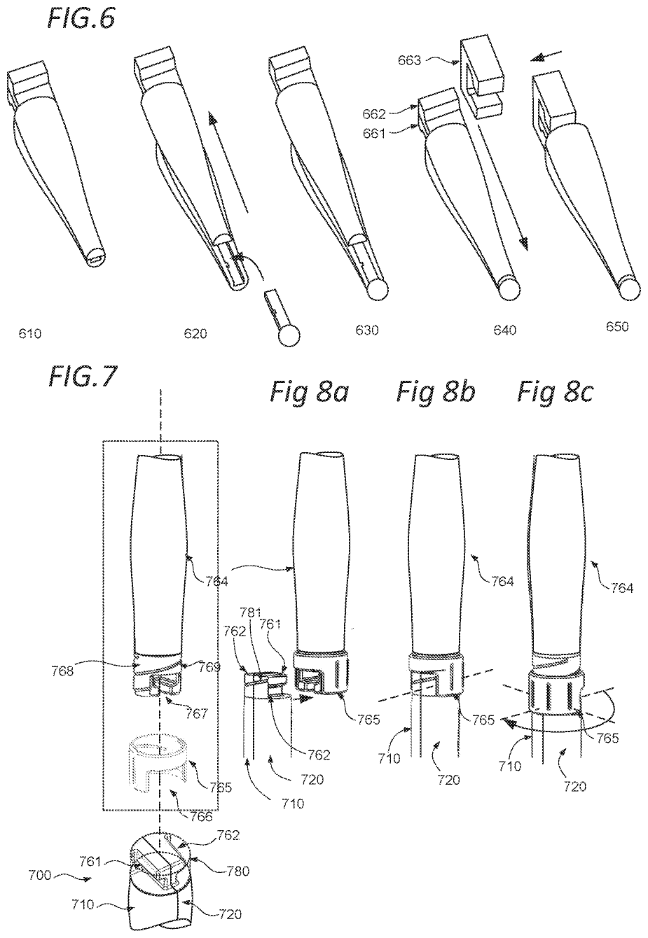

FIG. 6 illustrates a mode of operation of a tool holder in accordance with certain embodiments.

FIG. 6 shows four successive configurations in a mode of operation based on a tool holder as described above. The first view 610 shows the two slidabably mating parts aligned in the second configuration as described above.

The second view 620 shows the two slidabably mating parts separated in the first configuration as described above. This arrangement is accordingly achieved by sliding the upper of the two slidably mating parts backwards relative to the lower of the two slidably mating parts, to the limit of the extent permitted by the mutually engaging retaining elements, exposing the excavation in the lower slidabably mating part, so that the tool element may be inserted as discussed above.

The third view 630 shows the two slidabably mating parts separated in the first configuration as described above, with the tool element placed in the excavation.

The fourth view 640 shows the two slidabably mating parts aligned once more in the second configuration as described above. This arrangement is accordingly achieved by sliding the upper of the two slidably mating parts forwards relative to the lower of the two slidably mating parts, to the limit of the extent permitted by the mutually engaging retaining elements, enclosing the tool element between the two slidabably mating parts as discussed above.

On the strength of the foregoing, it will be appreciated that when the slidabably mating parts are aligned in the second configuration, if relative movement of the slidabably mating parts is prevented the tool is effectively locked in place.

In accordance with certain embodiments, each slidably mating part may comprise a respective locking element at a proximal extremity such that when the second slidably mating part is slid so as to bring the distal extremity of the second slidably mating part into proximity with the distal extremity of the second slidably mating part, i.e. the second configuration as described above, the respective locking elements align to form a keyed element. This keyed element may be inserted into a corresponding keyed channel of a restraining element so as to prevent further relative sliding movement of the slidably mating parts.

This is illustrated in FIG. 6. Specifically, as shown, each slidably mating part comprises a respective locking element 661, 662 at the proximal extremity, which in view 640 are aligned to form a keyed element. A schematic restraining element 663 is shown with a corresponding keyed channel. In view 650, the keyed element formed by the respective locking element 661, 662 has been inserted laterally into the keyed channel of the restraining element 663. It will be appreciated with the restraining element 663 now in position as shown in view 650 it is impossible for the one slidably mating part to move relative to the other, being constrained in all directions besides the longitudinal axis by the mutually engaging retaining elements, and prevented from relative movement in the longitudinal axis by the restraining element 663. As such, the tool element can be locked in place with no risk of inadvertent release.

The restraining element 663 can take any form. In particular, the restraining element 663 may conveniently take the form of a handle extension of the tool holder, so that in use the restraining element and tool holder may be geometrically continuous, so that the complete tool comprising the restraining element, tool holder and tool are substantially indistinguishable from a one piece tool.

According to certain variants, the dimensions of the respective locking elements may vary from front to back so that the force required to slide the keyed element formed thereby into the keyed channel of the restraining element increases as the keyed element of the tool progresses into the slot. In this case, once the keyed element is inserted in the keyed channel of the restraining element, the friction between the elements will tend to reduce the likelihood of the keyed element slipping back out without a deliberate application of force.

FIG. 7 shows a further development of locking mechanism introduced with reference to FIG. 6.

As shown in FIG. 7, a tool holder 700 generally as described above is provided whereby each slidably mating part 710mm 720 comprises a respective locking element 761, 762 at a proximal extremity thereof so formed that when the second slidably mating part 762 is slid so as to bring the distal extremity of the second slidably mating part into proximity with the distal extremity of the first slidably mating part 761, i.e. the second configuration as described above, the respective locking elements align to form a body whose outer extremities conform to a bounding cylinder 780.

Furthermore, in accordance with this development, the locking elements 761, 762 are provided with curved features 781 such that when the second slidably mating part 720 is slid so as to bring the distal extremity of the second slidably mating part 720 into proximity with the distal extremity of the first slidably mating part 710, the curved features elements 781 align to define at least part of one or more helical features coaxial with the bounding cylinder 780, such that a threaded ring 765 may by screwed onto tool holder.

Where a plurality of helical threads are provided, one or more of these threads may have dimensions different to other threads, such that the ring can only engage the cylindrical member in the desired orientation, for example so as to ensure proper positioning of the optional detent with respect to the slot in the extended and retracted positions. For example, the helical thread has a first root to crest ratio, and the second helical thread has a second root to crest ratio, wherein the threads of the ring are defined so that the ring can only engage the corresponding respective helical thread, and thereby may only be mounted on the handle in one configuration. Similarly, the circumferential spacing of the threads may be varied to achieve the same effect.

Correspondingly, a restraining element as described above may conform to the same bounding cylinder, as represented by the substantially cylindrical body 768, and comprise corresponding helical features 769 coaxial with the bounding cylinder, so that when the keyed element formed by the tool holder is inserted into the restraining element as described with reference to FIG. 6, a continuous threaded body is formed by the threads 762, 761 and 769, about which the threated ring 765 may be rotated to move between a retracted position where the keyed element may be freely inserted into or removed from the keyed channel 767, and an extended position in which the threaded ring traps the keyed element in the restraining element.

The pitch, the minor diameter, the major diameter, or the root to crest ratio of the external helical features 762, 761 and as the case may be 769 may vary along the length of the external helical features so that the force required to rotate a threaded ring increases as the ring is rotated about the proximal end of the tool holder from a retracted position to an extended position.

The threads on the cylindrical body 768 and/or the ring need not be continuous.

Besides the tool holder as described above, other embodiments may comprise a modular tool kit comprising a tool holder as described above, and additionally a handle 764 (corresponding for example to element 663 presented above) comprising the keyed channel, whereby the handle constitutes the restraining element so as to prevent further relative sliding movement of the slidably mating parts.

Accordingly In certain variants, the handle 764 may comprises a distal extremity 768 whose outer dimensions conform to a bounding cylinder bearing one or more helical features coaxial with the bounding cylinder, such that a threaded ring may by screwed onto the body.

In certain variants, the handle may comprise a distal extremity whose outer dimensions conform to a bounding cylinder bearing one or more helical features coaxial with the bounding cylinder, and the modular tool kit may further comprise a threaded ring that may by screwed onto the handle when engaged with the helical features.

In certain variants, the helical features of the handle 764 may permit an angular displacement of the threaded ring when rotated between an extended position and a retracted position is between 170 and 190 degrees.

As shown, the ring is provided with an optional detent 766 in the distal circumferential edge thereof. That is to say, the threaded ring may comprise an indentation at the distal edge thereof, the indentation being positioned on the circumference of the ring such that when the ring is in the retracted position the indentation is aligned with the slot.

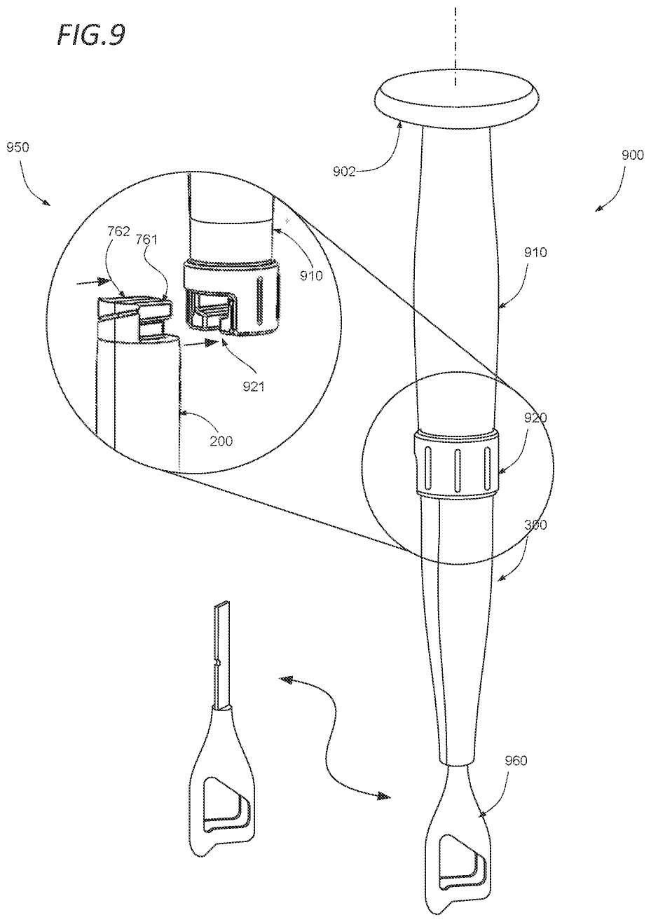

FIGS. 8a, 8b and 8c show the handle of FIG. 7 in different configurations. As shown, the threaded ring 765 is rotatable about the distal end of the handle 764 from a first, retracted position as shown in FIGS. 8a and 8b, to a second, extended as shown in FIG. 8c with helical features corresponding to those present in the keyed element formed by the respective locking elements 761, 762 of the slidably mating parts forming the tool holder as discussed above.

Corresponding to the helical features present in the keyed element formed by the respective locking elements 761, 762 of the slidably mating parts forming the tool holder as discussed above, the handle may be provided with a second or further external helical threads parallel the first external helical thread, wherein the threads of the ring engage both the first external helical thread and the second external helical thread.

FIG. 8a shows the ring in a first, retracted position prior to the insertion of the keyed element formed by the respective locking elements 761, 762 of the slidably mating parts forming the tool holder as discussed above. The tool holder may enclose a tool part as discussed above, and thereby form a complete tool. For example, the tool may comprise a surgical instrument. More particularly for example, the tool may be for surgery of the orthopaedic surgery or bone traumatology. Still more particularly for example tool may comprise an osteotome, a rasp or reamer or impactor. Alternatively, the secondary element may comprise an intermediate adaptor between the handle and a further component, where the further component may comprise a tool as discussed above or otherwise.

As shown the ring 765 is in a retracted position in which the ring leaves the keyed channel in the handle opening on one periphery of the distal end unobstructed. By this means, the keyed element formed by the respective locking elements 761, 762 of the slidably mating parts forming the tool holder as discussed above of may be introduced into the keyed channel of the handle.

As shown, the optional detent 766 as described with reference to FIG. 7 for example, aligns with the keyed channel in the retracted position.

FIG. 8b shows the handle in a first, retracted position after insertion of the keyed element formed by the respective locking elements 761, 762 of the slidably mating parts forming the tool holder as discussed above.

Since the keyed channel widens from the distal end towards the proximal end of the handle, a correspondingly formed keyed element formed by the respective locking elements 761, 762 of the slidably mating parts forming the tool holder as discussed above may be slid into the slot laterally as shown in FIG. 8b, but once in position, will not be movable along the axis of the handle, i.e. towards or away from the distal end of the handle, but only back or forth along the axis of the slot.

FIG. 8c shows the handle in a second, extended position after insertion of the keyed element formed by the respective locking elements 761, 762 of the slidably mating parts forming the tool holder as discussed above. As shown in FIG. 8c, the ring 765 has been rotated about the handle and tool holder, so that through the engagement of the threads of the ring in the threads of the tool holder, and as the case may be, of the handle, this rotational movement has been translated into a linear movement towards the distal end of the handle and the tool holder, so that the ring obstructs the keyed channel on one periphery of the distal end of the handle.

As shown, this movement comprises a rotation of 180° clockwise about the axis of the handle when viewed down the length from the proximal to the distal end of the handle. The skilled person will appreciate that the pitch of the threads and the angle through which the ring 765 is rotated will determine the distance along handle that the ring 765 travels. On one hand it is desirable that the distance travelled should be as great as possible, to allow the use of a long keyed element keyed element formed by the respective locking elements 761, 762 of the slidably mating parts forming the tool holder as discussed above, providing a strong and rigid connection between the handle and tool holder. On the other hand, in use it will be desirable that the user be required to turn the circle through as small an angle as possible, so that the manipulation may be performed with the lowest possible demand on the user's dexterity. The chosen compromise between these considerations is defined by the pitch of the respective threads of the ring and the tool holder. On this basis, the angular displacement of the ring between the extended position and the retracted position is preferably less than 361º, more preferably less than 271º, more preferably less than 181º. A multiple of 90° may be advantageous in terms of being more intuitive to the user. The angular displacement of the ring between the extended position and the retracted position is preferably more than 89º. The chosen compromise between these considerations is defined by the pitch of the respective threads of the ring and the tool holder.

Additional constraints may occur through a desire to limit the likelihood of the ring moving along the tool holder under its own weight, or in response to an accidental or incidental pressure, which will tend to favour a tighter pitch, although these considerations may also be managed by selecting materials, surfaces and/or tolerances so that the friction between elements reduces the risk of such unwanted movement. On this basis, the threads of the ring and tool holder may be configured in accordance with an embodiment such that the ring is turned through substantially one single revolution or less between the extended position and the retracted position. In accordance with a further embodiment the ring is turned through substantially one half revolution or less between the extended position and the retracted position. In accordance with a further embodiment the ring is turned through substantially one quarter revolution or less between the extended position and the retracted position. In accordance with a further embodiment the ring is turned through substantially one third revolution or more between the extended position and the retracted position. Meanwhile, the threads of the ring and tool holder may be configured in accordance with an embodiment such that the ring moves along the axis of the tool holder a distance substantially equal to 2 cm or less between the extended position and the retracted position. In accordance with a further embodiment the ring moves along the axis of the tool holder a distance substantially equal to 1.5 cm or less between the extended position and the retracted position. In accordance with a further embodiment the ring moves along the axis of the tool holder a distance substantially equal to 1 cm or less between the extended position and the retracted position. The skilled person will appreciate that any combination of angle of rotation and linear displacement may be selected within these ranges, and indeed outside these ranges. It may be noted that the thread of the cylindrical member and ring of FIGS. 8a, 8b and 8c comprises a double thread. A double thread may be advantageous in providing a firm engagement between the threads of the ring and the cylinder while permitting a short threaded section along the length of the ring, so as to minimise the dimensions of the ring. The skilled person will appreciate that a single thread may also be adequate in many implementations. The skilled person will appreciate that a three or even more threads may be appropriate in other implementations.

As shown in FIGS. 7 and 8a, the keyed channel defines substantially a T cross section, and the keyed element formed by the respective locking elements 761, 762 of the slidably mating parts forming the tool holder as discussed above is shaped correspondingly. This shape achieves the objective of ensuring that the keyed element can only enter or exit the slot through the lateral opening of the cylindrical section, and that once the ring is in the extended position, no force in any direction on the secondary element with respect to the handle will separate the secondary element from the handle. The skilled person will recognise that the keyed element and corresponding slot may have any form complying with the general requirement that it widens from the distal end towards the proximal end. As such, it may form a wedge, dovetail or t section as described above. It may furthermore be circular, elliptical, rectangular, square, or any other form. The keyed element will generally constitute an extrusion of the chosen cross section from one side to the other. In certain embodiments, the keyed element may taper from one side to the other.

Where this is the case the slot may taper from side to side correspondingly. Where this is the case, the slot may be defined as being deeper from side to side that the length from side to side of the corresponding keyed element. On this basis, the tapering walls of the keyed element will engage the sides of the keyed element before the end of the keyed element reached the lateral extremity of the slot. By this means, the slot will become progressively tighter as the keyed element is inserted, and a firm insertion without any play between the handle and secondary element may be achieved by pushing the keyed element fully into the slot.

As such, as shown the keyed element of the tool holder is entirely trapped in the slot by the new position of the ring, as the result of a simple half turn (in the present example) of the ring, which may be effectuated by the user with a movement of the thumb, retaining the tool in the other fingers of the actuating hand, and leaving the other hand free. To further facilitate this action, the ring may be knurled, grooved, provided with a non-slip coating, provided with flats or otherwise treated to improve the users grip thereon. Where the ring is provided with flats, these may be even in number, and may further be dimensioned so that they may be engaged using a spanner of standard dimensions, for example as defined in ISO/TC 29/SC 10 and the like.

In certain embodiments, the shape or dimension of ring and cylindrical member, and in particular the thread on either or both of the ring or the cylindrical member may vary along their length, for example such that their engagement becomes progressively tighter towards the extended position, so that while it may move freely at certain portions of its travel, as it approaches the extended position it becomes somewhat resistant to movement. This approach may be advantageous in further reducing the risk of the ring being moved from the extended position inadvertently, which might otherwise lead to a loosening or even decoupling of the secondary element from the handle. Similarly, the engagement between the ring and cylindrical member may become progressively tighter towards the retracted position, so that while it may move freely at certain portions of its travel, as it approaches the retracted position it becomes somewhat resistant to movement. This approach may be advantageous in further reducing the risk of the ring being moved from the retracted position inadvertently, which might otherwise complicate the task of inserting the keyed element of the secondary element. The variations in shape or dimensions may include for example variations in the pitch, the minor diameter, the major diameter, or the root to crest ratio or any combination of these. Additionally or alternatively a similar effect may be achieved by a variation in the outer diameter of the cylindrical member and/or the inner diameter of the ring along the length thereof, for example so that as the ring moves towards the extended position, it becomes progressively tighter on the cylindrical member and/or so that as the ring moves towards the retracted position, it becomes progressively tighter on the cylindrical member.

As discussed above, the slot (and correspondingly the keyed element) widens from the distal end towards the proximal end. As discussed above, the slot, and correspondingly the keyed element may take many forms whilst satisfying this requirement.

FIG. 9 shows an assembled tool in accordance with an embodiment.

As shown, there is provided a modular tool 900 comprising a handle 910, and a tool holder 300 substantially as described above. The handle 910 comprises a releasable coupling comprising by way of example a threaded ring 920, the threads of the ring engaging an external helical thread of the cylindrical member of the handle 910 and tool holder 300. The threaded ring 920 is rotatable about the distal end of the member between an extended position as shown in FIG. 8c which the ring obstructs a slot opening on one periphery of the distal end of the handle, and a retracted position as shown in the emphasised section 950 in which the ring leaves the slot 921 opening on one periphery of the distal end of the body unobstructed, into which a keyed element formed by the respective locking elements 761, 762 of the slidably mating parts forming the tool holder as discussed above may be inserted. When the ring is rotated about the cylindrical member, it progressing along the length thereof and whilst progressing lengthwise and rotating, obstructs a slot 921 as shown in FIG. 8c to trap the tool holder in place.

The handle may optionally be provided with an angle datum such as a radial line on the guard plate, or a radial lumen through which a bar may by inserted.

As such the tool holder may further comprise a tongue element provided at a proximal end thereof, the tongue being widening from its junction with the tool holder.

The tool holder encloses a tool element 960 substantially as described for example with reference to FIGS. 3 to 5. Specifically, as shown the tool element is an osteotome, as may be used for example in surgery of the hip.

As shown, the handle further comprises an optional guard plate 902 at the proximal end thereof. Such a guard plate may serve to protect the hand of a user when gripping the handle 920 from blows struck against the proximal end thereof with a hammer, mallet or the like, for example where the tool or working part 960 is a chisel, reamer or other such tool requiring a percussive application.

One field in which a handle as described may be appropriate is that of surgical instruments, such that the modular tool as a whole may comprise or constitutes a surgical instrument. More particularly, the modular tool may be for orthopaedic surgery or bone traumatology. More particularly, the modular tool may be for surgery of the hip, shoulder or knee. More particularly, as shown, the working part 960, and thus the modular tool as a whole 900 comprises a box osteotome. It will be appreciated that in line with the many fields of application and associated tool types that may be envisaged, many different possible working parts 960 may be envisaged, for use with a single handle in accordance with embodiments as described above. Further examples of possible working parts, and resulting modular tools, include an osteotome, impactor and many other tools as will readily occur to the skilled person.

The tool holder of the present invention may be formed of any material. In particular, it may be formed of steel, aluminium, titanium or any other suitable metal or alloy. It may also be formed of a thermoplastic or other synthetic material. It may in particular be formed from a polyamide, for example a polyarylamide. The synthetic material may comprise additional components such as a filler, swelling agent and the like. It may still further be formed of a synthetic composite material, comprising a glass, carbon fibre, carbon nanoparticle or any other material exhibiting a high tensile strength, in a matrix of a synthetic material, such as any of those listed above. In certain embodiments, the tool holder may be composed of a glass fibre reinforced polyarylamide, such as for example that marketed by the Solvay corporation under the trademark “Ixef GS 1022”.

The tool holder may be formed of different materials in different regions, including metal parts and synthetic parts. The handle may also comprise voids for the purpose of economy of material, reduced weight and so on.

Where the tool holder is incorporated in a modular tool as shown in FIG. 8, the too part 960 and/or handle 920 may each be composed of the materials mentioned above. In some embodiments, the handle, tool holder and working part may all be composed of the same material.

Accordingly, there is provided a tool holder for a modular tool in which a tool element is trapped between two slidably mating parts, which may be slidingly aligned to trap the tool element, and slidingly separated to free the tool. The sliding operation of the slidably mating parts is defined by mutually engaging retaining elements configured to allow relative movement of said slidably mating parts along said longitudinal axis, but prevent relative movement in any other direction. The tool holder may form part of a kit comprising one or more tool holders, one or more tool elements, and one or more handles. The handle may be comprised with key elements engaging a proximal extremity of the slidably mating parts when aligned, to that with the handle in position, the slidably mating parts cannot be separated, and the tool element released.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A tool holder for a modular tool, said tool holder having a distal end and a proximal end disposed on a longitudinal axis, said tool holder for a modular tool being split longitudinally into two slidably mating parts, said slidably mating parts being provided with mutually engaging retaining elements, said mutually engaging retaining elements being configured to allow relative movement of said slidably mating parts along said longitudinal axis, but prevent relative movement in any other direction, a first said slidably mating part being provided with a longitudinal excavation extending from the distal end thereof such that said longitudinal excavation may be obturated but for the distal extremity thereof by sliding the second slidably mating part so as to bring the distal extremity of the second slidably mating part into proximity with the distal extremity of the first slidably mating part, and such that said longitudinal excavation may be exposed by sliding the second slidably mating part so as to separate the distal extremity of the second slidably mating part from the distal extremity of the first slidably mating part, whereby a tool element may be inserted into said excavation when exposed, and trapped therein when obturated.

2. The tool holder of claim 1, wherein each said slidably mating part comprises a respective locking element at a proximal extremity thereof such that when the second slidably mating part is slid so as to bring the distal extremity of the second slidably mating part into proximity with the distal extremity of the first slidably mating part, said respective locking elements align to form a keyed element, whereby said keyed element may be inserted into a corresponding channel of a restraining element so as to prevent further relative sliding movement of said slidably mating parts.

3. The tool holder of claim 1 wherein each said slidably mating part comprises a respective locking element at a proximal extremity thereof such that when the second slidably mating part is slid so as to bring the distal extremity of the second slidably mating part into proximity with the distal extremity of the first slidably mating part, said respective locking elements align to form a body whose outer extremities conform to a bounding cylinder, said locking elements being provided with curved features such that when the second slidably mating part is slid so as to bring the distal extremity of the second slidably mating part into proximity with the distal extremity of the first slidably mating part, said curved features elements align to define at least part of one or more helical features coaxial with said bounding cylinder, such that a threaded ring may by screwed onto said tool holder.

4. The tool holder of claim 1, said tool holder being composed of a synthetic material or a synthetic composite material.

5. A modular tool kit comprising a tool holder according to claim 1, and a handle comprising said keyed channel, whereby said handle constitutes said restraining element so as to prevent further relative sliding movement of said slidably mating parts.

6. The modular tool kit of claim 5, wherein said handle comprises a distal extremity whose outer dimensions conform to a bounding cylinder bearing one or more helical features coaxial with said bounding cylinder, such that a threaded ring may by screwed onto said body.

7. The modular tool kit of claim 6, wherein said handle comprises a distal extremity whose outer dimensions conform to a bounding cylinder bearing one or more helical features coaxial with said bounding cylinder, said modular tool kit further comprising a threaded ring that may by screwed onto said handle when engaged with said helical features.

8. The modular tool kit of claim 1 further comprising a tool element adapted to be inserted into said excavation when exposed, and trapped therein when obturated.

9. A surgical instrument constituted by a modular tool kit of claim 7.

10. A surgical instrument for orthopaedic surgery or bone traumatology constituted by a modular kit of claim 7.

11. An osteotome constituted by a modular tool kit of claim 7.

12. An impactor for implants constituted by a modular tool kit of claim 7.
